SEQUENCE LISTING (4 sequences)

SEQ ID NO: 1 - DNA, 2691 bp, Homo sapiens
gcttgcccgtcggtcgctagctcgctcggtgcgcgtcgtcccgctccatggcgctcttcgtgcggctgctggctctcgccctggctctggccctgggccccgccgcgaccctggcgggtcccgccaagtcgccctaccagctggtgctgcagcacagcaggctccggggccgccagcacggccccaacgtgtgtgctgtgcagaaggttattggcactaataggaagtacttcaccaactgcaagcagtggtaccaaaggaaaatctgtggcaaatcaacagtcatcagctacgagtgctgtcctggatatgaaaaggtccctggggagaagggctgtccagcagccctaccactctcaaacctttacgagaccctgggagtcgttggatccaccaccactcagctgtacacggaccgcacggagaagctgaggcctgagatggaggggcccggcagcttcaccatcttcgcccctagcaacgaggcctgggcctccttgccagctgaagtgctggactccctggtcagcaatgtcaacattgagctgctcaatgccctccgctaccatatggtgggcaggcgagtcctgactgatgagctgaaacacggcatgaccctcacctctatgtaccagaattccaacatccagatccaccactatcctaatgggattgtaactgtgaactgtgcccggctcctgaaagccgaccaccatgcaaccaacggggtggtgcacctcatcgataaggtcatctccaccatcaccaacaacatccagcagatcattgagatcgaggacacctttgagacccttcgggctgctgtggctgcatcagggctcaacacgatgcttgaaggtaacggccagtacacgcttttggccccgaccaatgaggccttcgagaagatccctagtgagactttgaaccgtatcctgggcgacccagaagccctgagagacctgctgaacaaccacatcttgaagtcagctatgtgtgctgaagccatcgttgcggggctgtctgtagagaccctggagggcacgacactggaggtgggctgcagcggggacatgctcactatcaacgggaaggcgatcatctccaataaagacatcctagccaccaacggggtgatccactacattgatgagctactcatcccagactcagccaagacactatttgaattggctgcagagtctgatgtgtccacagccattgaccttttcagacaagccggcctcggcaatcatctctctggaagtgagcggttgaccctcctggctcccctgaattctgtattcaaagatggaacccctccaattgatgcccatacaaggaatttgcttcggaaccacataattaaagaccagctggcctctaagtatctgtaccatggacagaccctggaaactctgggcggcaaaaaactgagagtttttgtttatcgtaatagcctctgcattgagaacagctgcatcgcggcccacgacaagagggggaggtacgggaccctgttcacgatggaccgggtgctgacccccccaatggggactgtcatggatgtcctgaagggagacaatcgctttagcatgctggtagctgccatccagtctgcaggactgacggagaccctcaaccgggaaggagtctacacagtctttgctcccacaaatgaagccttccgagccctgccaccaagagaacggagcagactcttgggagatgccaaggaacttgccaacatcctgaaataccacattggtgatgaaatcctggttagcggaggcatcggggccctggtgcggctaaagtctctccaaggtgacaagctggaagtcagcttgaaaaacaatgtggtgagtgtcaacaaggagcctgttgccgagcctgacatcatggccacaaatggcgtggtccatgtcatcaccaatgttctgcagcctccagccaacagacctcaggaaagaggggatgaacttgcagactctgcgcttgagatcttcaaacaagcatcagcgttttccagggcttcccagaggtctgtgcgactagcccctgtctatcaaaagttattagagaggatgaagcattagcttgaagcactacaggaggaatgcaccacggcagctctccgccaatttctctcagatttccacagagactgtttgaatgttttcaaaaccaagtatcacactttaatgtacatgggccgcaccataatgagatgtgagccttgtgcatgtgggggaggagggagagagatgtactttttaaatcatgttccccctaaacatggctgttaacccactgcatgcagaaacttggatgtcactgcctgacattcacttccagagaggacctatcccaaatgtggaattgactgcctatgccaagtccctggaaaaggagcttcagtattgtggggctcataaaacatgaatcaagcaatccagcctcatgggaagtcctggcacagtttttgtaaagcccttgcacagctggagaaatggcatcattataagctatgagttgaaatgttctgtcaaatgtgtctcacatctacacgtggcttggaggcttttatggggccctgtccaggtagaaaagaaatggtatgtagagcttagatttccctattgtgacagagccatggtgtgtttgtaataataaaaccaaagaaacata

SEQ ID NO: 2 - protein, 683 aa, Homo sapiens
MALFVRLLALALALALGPAATLAGPAKSPYQLVLQHSRLRGRQHGPNVCAVQKVIGTNRKYFTNCKQWYQRKICGKSTVISYECCPGYEKVPGEKGCPAALPLSNLYETLGVVGSTTTQLYTDRTEKLRPEMEGPGSFTIFAPSNEAWASLPAEVLDSLVSNVNIELLNALRYHMVGRRVLTDELKHGMTLTSMYQNSNIQIHHYPNGIVTVNCARLLKADHHATNGVVHLIDKVISTITNNIQQIIEIEDTFETLRAAVAASGLNTMLEGNGQYTLLAPTNEAFEKIPSETLNRILGDPEALRDLLNNHILKSAMCAEAIVAGLSVETLEGTTLEVGCSGDMLTINGKAIISNKDILATNGVIHYIDELLIPDSAKTLFELAAESDVSTAIDLFRQAGLGNHLSGSERLTLLAPLNSVFKDGTPPIDAHTRNLLRNHIIKDQLASKYLYHGQTLETLGGKKLRVFVYRNSLCIENSCIAAHDKRGRYGTLFTMDRVLTPPMGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQPPANRPQERGDELADSALEIFKQASAFSRASQRSVRLAPVYQKLLERMKH

SEQ ID NO: 3 - DNA, 20 bp, Artificial Sequence
  synthetic oligonucleotide
gaagtcctggactccctggt

SEQ ID NO: 4 - DNA, 20 bp, Artificial Sequence
  synthetic oligonucleotide
ctgcagcccacctccagtgt

(19) United States
(12) Patent Application Publication
(10) Pub. No.: US 2005/0244345 A1
(43) Pub. Date: Nov. 3, 2005

(54) Method for preventing mineralization in the periodontal ligament (PDL)

(75) Inventors: Shigeru Ohno, Hiroshima-shi (JP); Kazuo Tanne, Hiroshima-shi (JP); Takeyoshi Doi, Hiroshima-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: President of Hiroshima University, Higashihiroshima-shi (JP)

(21) Appl. No.: 11/081,566

(22) Filed: Mar. 17, 2005

Related U.S. Application Data
(62) Division of application No. 10/717,708, filed on Nov. 21, 2003.

(51) Int. Cl. A61K 38/17; A61K 7/28

(52) U.S. Cl. 424/50; 514/12

(57) ABSTRACT

The present invention provides an agent containing RGD-CAP for of suppressing mineralization of the periodontal ligament and preventing the adhesion of teeth. The present invention provides the method of suppressing mineralization in the periodontal ligament and preventing the adhesion of teeth.

### BACKGROUND OF THE INVENTION

1. Field of the Invention

The invention relates to a method for preventing mineralization in the periodontal ligament (PDL).

2. Description of the Related Art

In the dental clinical field, externally damaged teeth and mal-transplantation of teeth cannot be maintained. This is mostly because the periodontal ligament degenerates, causing bone adhesion. However, any effective treatment has not been found for preventing or treating such bone adhesion. Many proteins are known to be involved in mineralization of the periodontal ligament tissues; however factors for suppressing the mineralization have not been elucidated.

In previous studies, we isolated a collagen-associated protein containing the RGD (arginine-glycine-aspartic acid) sequence named RGD-CAP from a collagen fiber-rich fraction of cartilage, and demonstrated that this protein binds to collagens and is identical to human βig-h3 (Hashimoto K, Noshiro M, Ohno S, Kawamoto T, Satakeda H, Akagawa Y, et al. (1997). Characterization of a cartilage-derived 66-kDa protein (RGD-CAP/beta ig-h3) that binds to collagen. Biochim Biophys Acta 1355: 303-314).

The characteristic four repetitive structures similar to RGD-CAP/βig-h3 without the RGD motif were found in insect fasciclin I, as well as osteoblast specific factor 2 (OSF-2)/periostin (Skonier J, Neubauer M, Madisen L, Bennett K, Plowman G D, Purchio A F (1992). cDNA cloning and sequence analysis of beta ig-h3, a novel gene induced in a human adenocarcinoma cell line after treatment with transforming growth factor-beta. DNA Cell Biol 11: 511-522.; Wang W C, Zinn K, Bjorkman P J (1993). Expression and structural studies of fasciclin I, an insect cell adhesion molecule. J Biol Chem 268: 1448-1455; Horiuchi K, Amizuka N, Takeshita S, Takamatsu H, Katsuura M, Ozawa H, et al. (1999). Identification and characterization of a novel protein, periostin, with restricted expression to periosteum and periodontal ligament and increased expression by transforming growth factor beta. J Bone Miner Res 14: 1239-1249). These proteins were also shown to have similar functions in cell adhesion (Takeshita S, Kikuno R, Tezuka K, Amann E (1993). Osteoblast-specific factor 2: cloning of a putative bone adhesion protein with homology with the insect protein fasciclin I. Biochem J 294: 271-278; Sugiura T, Takamatsu H, Kudo A, Amann E (1995). Expression and characterization of murine osteoblast-specific factor 2 (OSF-2) in a baculovirus expression system. Protein Expr Purif 6: 305-311.; Ohno S, Noshiro M, Makihira S, Kawamoto T, Shen M, Yan W, et al. (1999). RGD-CAP ((beta)ig-h3) enhances the spreading of chondrocytes and fibroblasts via integrin alpha(1)beta(1). Biochim Biophys Acta 1451: 196-205.; Horiuchi et al., 1999), and have been categorized as the fasciclin family.

Recently, it has been demonstrated that the mRNA level of RGD-CAP/βig-h3 was decreased in human bone marrow stromal cells (BMSC) treated with Dex, which promotes osteogenic differentiation of BMSC (Dieudonné S C, Kerr J M, Xu T, Sommer B, DeRubeis A R, Kuznetsov S A, et al. (1999). Differential display of human marrow stromal cells reveals unique mRNA expression patterns in response to dexamethasone. J Cell Biochem 76: 231-243.), and that RGD-CAP/βig-h3 inhibited bone nodule formation of mouse osteoblasts in vitro (Kim J E, Kim E H, Han E H, Park R W, Park I H, Jun S H, et al. (2000). A TGF-beta-inducible cell adhesion molecule, betaig-h3, is downregulated in melorheostosis and involved in osteogenesis. J Cell Biochem 77: 169-178.). Furthermore, our recent studies have shown that recombinant RGD-CAP inhibited the mineralization of hypertrophic chondrocytes (Ohno S, Doi T, Tsutsumi S, Okada Y, Yoneno K. Kato Y et al. (2002). RGD-CAP (βig-h3) is expressed in precartilage condensation and in prehypertrophic chondrocytes during cartilage development. Biochim Biophys Acta 1572: 114). These findings indicate that RGD-CAP/βig-h3 functions as a negative regulator of osteogenesis.

Lately, we have reported that RGD-CAP is expressed also in the human periodontal ligament. We also found that the expression of RGD-CAP is enhanced by applying a mechanical load to the periodontal ligament cells (Doi T et al. (2001). Journal of Dental Research vol. 80 Special Issue (IADR abstract), p 783). This document, however only suggests that RGD-CAP has a function of suppressing the alkaline phosphatase activity. The function of RGD-CAP specific to the periodontal ligament cells still remains unknown. More specifically, whether RGD-CAP is involved in mineralization of the periodontal ligament cells or not is not clear.

### BRIEF SUMMARY OF THE INVENTION

According to an aspect of the present invention, there is provided an agent for suppressing mineralization in the periodontal ligament and an agent for preventing adhesion of teeth.

According to another aspect of the present invention, there is provided a method for suppressing mineralization in the periodontal ligament and preventing adhesion of teeth.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate presently preferred embodiments of the invention, and together with the general description given above and the detailed description of the preferred embodiments given below, serve to explain the principles of the invention.

FIG. 1 shows Expression of RGD-CAP in the PDL. Western blot analysis of RGD-CAP in human PDL in the presence of β-mercaptoethanol (β-ME; 0-5.0%);

FIGS. 2A to 2C show RGD-CAP mRNA level and ALP activity cultured PDL cells. After the PDL cells became confluent, the cells became confluent, the cells maintained in Medium A were treated with 10-8 M dexamethasone (Dex) or 10-8 M 1α,25-dihydroxyvitamin D₃ (Vitamin D₃) for 0-11 days. The rate of increase in RGD-CAP mRNA expression determined by real-time PCR is shown as a bar. The dotted line indicates the level of RGD-CAP mRNA on day 0. Levels of ALP activity in these cultures were evaluated by measurement of the absorbance at 405 nm and are shown as lines. Values are averages ±SD of triplicate cultures; and

FIGS. 3A to 3C show effects of RGD-CAP on mineralization of PDL cells. (A) The PDL cells were seeded confluently on the 35-mm dishes coated with recombinant RGD-CAP (20 μg/ml), maintained in medium A for 1-5 days. ALP activity was measured following the method described in MATERIALS & METHODS. Values are average values are averages ±SD of triplicate cultures. (B, C) A 20-μg/mL quantity of recombinant RGD-CAP in the solution buffer (PBS containing 4 M urea) or solution buffer (control) was added to the mineralizing medium (MM) of PDL cell cultures every 2 days. Total RNA was extracted on day 11, and RT-PCR was performed. Ethidium bromide staining pattern of PCR products of type I collagen (Col I), bone sialoprotein (BSP), and glyceradehyde-3-phosphate dehydrogenase (G3PDH) (B). (AQ) We determined the relative mRNA expression of Col I or BSP by dividing the densitomeric value of RT-PCR products of each transcript by that of G3PDH. Alizarin red staining was performed for those (AQ) cultured on day 21, and the number of bone nodules was counted (C). ** p<0.01.

### DETAILED DESCRIPTION OF THE INVENTION

Now, embodiments of the present invention will be explained below.

In an aspect of the present invention, there is provided an agent for suppressing mineralization of the periodontal ligament and an agent for preventing adhesion of teeth.

The agent for suppressing mineralization of the periodontal ligament and the agent for preventing adhesion of teeth according to the present invention contain RGD-CAP. RGD-CAP to be used in the present invention may be derived from any species, however, human RGD-CAP (encoded by a gene deposited at the GenBank under accession No. NM_{—}000358) is preferable. The protein used in the present invention includes a protein having the same amino acid sequence as that of the RGD-CAP and substantially the same amino acid sequence as that of the RGD-CAP.

As the protein having substantially the same amino is acid sequence as that of the RGD-CAP, refers to proteins containing about 70% or more of homologous amino acid sequence to that of the RGD-CAP, preferably about 80% or more, more preferably about 90% or more, the most preferably, about 95%. The protein having substantially the same amino acid sequence as the amino acid sequence of the RGD-CAP is preferably a protein having substantially the same amino acid sequence as the aforementioned amino acid sequence and substantially the same activity as the protein having the amino acid sequence of RGD-CAP.

As substantially the same activity, an alkaline phosphatase suppressing activity and a mineralization suppressing activity, and small bone node suppressing activity may be mentioned.

The “substantially the same activity”, means that activities are physiochemically or pharmacologically the same. To determining whether a protein has such an activity or not, but not limited, the methods shown in Examples below may be used.

Furthermore, examples of RGD-CAP includes
proteins having the amino acid sequence of RGD-CAP from which one or a plurality of amino acids (preferably about 1 to 7, more preferably about 1 to 5, most preferably 1 to 3) have been deleted;
proteins having the amino acid sequence of RGD-CAP to which one or plurality of amino acids (preferably about 1 to 7, more preferably about 1 to 5, most preferably 1 to 3) have been added or inserted; and/or
proteins having the amino acid sequence of RGD-CAP having one or amino acids (preferably about 1 to 7, more preferably about 1 to 5, most preferably 1 to 3) replaced with other amino acids.

As RGD-CAP, proteins having the aforementioned amino acid sequences singly or in combination may be used.

The RGD-CAP used in the present invention can be obtained by purifying from (the protein produced by) a transformant having a gene encoding RGD-CAP. The gene encoding RGD-CAP (for example, cDNA) can be obtained by the following methods.

It has been known that RGD-CAP is expressed more or less in various types of cells. From these cells, for example cultured PDL cells, the total RNA is purified using a commercially available RNA extraction kit. As another method for purifying total RNA, first, PDL cells are homogenized in phenol or a phenol chloroform solution containing guanidine isothiocyanate, and centrifugally separated into a water phase and an organic phase. The obtained water phase is added to isopropanol to precipitate, thereby recovering the total RNA. Alternatively, the total RNA can be recovered by sugar or cesium chloride density gradient centrifugation. Using the total RNA as a template and oligo (dT) as a primer, cDNA is synthesized from mRNA (i.e., poly(A)RNA) with a reverse transcriptase.

To ligate cDNA to a phage or a plasmid vector, an appropriate restriction site is prepared previously and the cDNA may be ligated to the same restriction site of a phage or a plasmid vector. The vector obtained may be transduced or transfected to Escherichia coli to construct a cDNA library. Alternatively, commercially available cDNA library derived from the total RNA obtained form various types of cells may be used.

Since the aforementioned cDNA (cDNA library) contains DNA fragments having different information items other than a target DNA fragment encoding RGD-CAP, only the target DNA must be amplified. Since the sequence of RGD-CAP gene has been known, the target DNA encoding RGD-CAP can be exclusively amplified by designing a primer based on the sequence of a RGD-CAP gene and performing a PCR reaction by using the cDNA library as a template. More specifically, when human RGD-CAP cDNA is amplified, a complementary DNA sequence to the sequence of human RGD-CAP cDNA is prepared as a primer (for example, a forward primer: GAAGTCCTGGACTCCCTGGT, a reverse primer CTGCAGCCCACCTCCAGTGT) based on the sequence, and PCR is performed by using the aforementioned cDNA library as a template. In this manner, RGD-CAP cDNA can be specifically amplified.

After the target RGD-CAP cDNA is cloned and amplified, the cDNA is recovered and electrophoretically purified. The obtained cDNA may be identified by sequencing or the like.

Subsequently, the purified cDNA is ligated downstream of the promoter of an appropriate expression vector. As the expression vector, a plasmid derived from Escherichia coli, such as pET28a, pBR322, pBR325 or pUC12 may be used.

The expression vector obtained is introduced into an appropriate host cell and expressed.

More specifically, RGD-CAP cDNA is introduced into a vector for Escherichia coli as described in Examples below and the vector is introduced into Escherichia coli to express RGD-CAP. Alternatively, RGD-CAP may be purified via the inclusion body of RGD-CAP.

Examples of other expression vectors include a plasmid derived from Bacillus subtilis, a plasmid derived from yeast, animal viruses such as retrovirus, vaccinia virus, and Baculovirus, and vectors for mammals. Any promoter may be used as long as it is an appropriate promoter suitable for a host for use in gene expression. The expression vectors, if desired, may contain an enhancer, splicing signal, poly-A addition signal, selection markers such as Ampiciline resistant gene, and signal sequences.

As a host to which an expression vector is introduced varies depending upon the expression vector to be used. For example, Escherichia coli, yeasts, insect cells, and animal cells may be used.

After incubation, bacterial cells are collected by a known method and suspended in an appropriate buffer. Subsequently, the bacteria cells are broken by ultrasonic wave, lysozyme, and/or freeze/thaw treatment and subjected to centrifugal separation and filtration to obtain a crude solution of a peptide extract. The buffer may contain a peptide denaturing agent such as urea and guanidine chloride, and a surfactant such as Triton X-100 (Sigma, Chemical Co., Ltd., St. Louis, USA). When RGD-CAP is secreted into the medium, the supernatant may be collected by separating it from the bacteria cells by a known method after culturing. The RGD-CAP contained in the supernatant thus obtained or the extraction solution is purified by known separation and purification methods in appropriate combination. Examples of the known separation and purification methods include methods based on difference in solubility such as salting-out and solvent precipitation; methods based on difference in molecular weight, such as dialysis, ultrafiltration, gel filtration, and SDS polyacrylamide gel electrophoresis; methods based on difference in charge such as ion exchange chromatography; method based on specific affinity, such as affinity chromatography; method based on hydrophobic/hydrophilic natures such as reverse-phase high performance liquid chromatography; and method based on difference in isoelectric point such as electrophoresis.

When an inclusion body is obtained by using a Escherichia coli expression vector as shown in Examples below, the inclusion body is dissolved with 7 M urea (Wako Pure Chemical Industries Ltd.), passed through 0.45 μm filter (Millipore, Bedford, Mass., USA), trapped by a His trap column (1 ml, Amersham Bioscience Corp, Piscataway, N.J., USA), and eluted with 10 ml of solution containing 20 mM sodium phosphate (pH 7.4), 0.5 M NaCl, and 10 mM imidazole, and further with 5 ml of solution containing 20 mM sodium phosphate, 0.5 M NaCl, and 500 mM imidazole, sequentially. Finally, 300 and 500 mM imidazole fractions were collected to purify RGD-CAP.

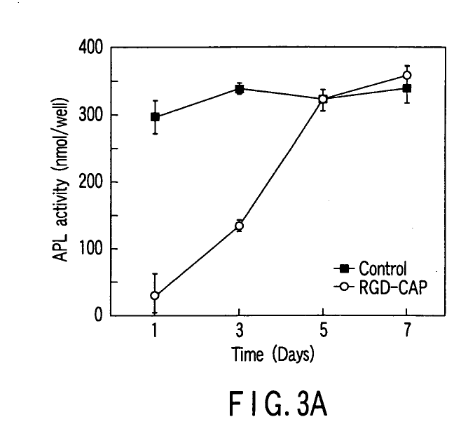

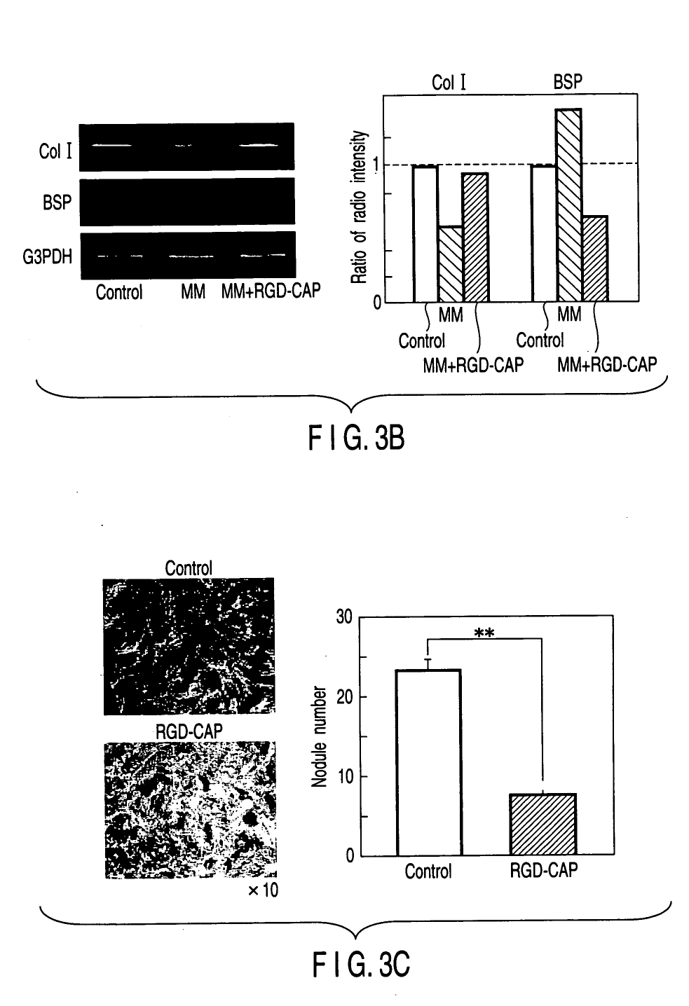

Then, a vector is constructed by inserting cDNA containing a total-length human RGD-CAP into an EcoRI site of a pET-28a protein expression vector (Novagen, Madison, USA) having T7 promoters (FIG. 3). After the orientation of the cDNA inserted is identified, the vector is introduced into E. coli HMS174DE3 (Novagen) to transform it and cultured in 30 μg/ml of liquid broth (LB) containing kanamycin (Meiji Seika Kaisha Ltd., Tokyo) at 37° C. When the absorbance of OD600 nm reaches 0.6, isopropyl-b-D(−)-thiogalactopyranoside (IPTG; Wako Pure Chemical Industries Ltd., Osaka) is added to the culture to make the final concentration of 1 mM and cultivation is continued for 3 hours. Thereafter, the bacterial cells are recovered by centrifuge, suspended in a solution containing 50 mM Tris-HCl (pH 8.0), 0.3 mg/ml of lysozyme, and 1 mM EDTA, and allowed to stand still on ice for 30 minutes. To the solution mixture, sodium deoxycholate (Wako Pure Chemical Industries Ltd.) is added to make the final concentration to 0.3% and allowed to stand still on ice for 20 minutes. Thereafter, bacterial cells are completely sonicated.

The precipitates obtained by centrifuge is suspended in a solution containing 50 mM Tris-HCl (pH 7.5), 0.5% Triton-X (Sigma, Chemical Co., Ltd., St. Louis, USA), 10 mM EDTA, and 0.1 M NaCl solution, and again separated by centrifuge. The resulting precipitate is dissolved in 7 M urea (Wako Pure Chemical Industries Ltd.), passed through a 0.45 μm filter (Millipore, Bedford, USA), trapped by a His trap column (volume 1 ml, Amersham Bioscience Corp., Piscataway, USA), eluted with 10 ml of solution containing 20 mM sodium phosphate (pH 7.4), and 0.5 M NaCl, 10 mM imidazole, and further with 5 ml of solution containing 20 mM sodium phosphate, 0.5 M NaCl, and 500 mM imidazole, sequentially. After fractions of 300 and 500 mM imidazole are collected, dialysis is performed against 2 l of 7 M urea for 2 days in order to remove imidazole. The purified recombinant RGD-CAP is passed through a 0.22 μm filter (Millipore) to sterile it.

RGD-CAP may be obtained either in liquid form containing RGD-CAP dissolved in an appropriate solution or powder form in the aforementioned manner. Alternatively, RGD-CAP may be formulated into a composition containing a pharmacologically acceptable solvent, excipient, carrier, auxiliary agent, and so forth, such as a liquid, lotion, aerosol, injection, powder, and granular agents, in accordance with a common method for producing a preparation. The content of an active ingredient, RGD-CAP, varies depending upon the form of a preparation, however, about 0.1 to 100 wt %, preferably about 10 to 100 wt %, and more preferably about 20 to 100 wt % may be used.

The agent for suppressing mineralization in the periodontal ligament and preventing adhesion of teeth may be used in transplanting a tooth to a patient. Since the mineralization of teeth in the periodontal ligament can be suppressed by using the agent for suppressing mineralization of the periodontal ligament and preventing adhesion of teeth, thereby preventing or inhibiting bone adhesion involving degeneration in the periodontal ligament. The agent of the present invention effectively prevents or inhibits bone adhesion even if it is applied to a tooth having external damage as well as a transplanted tooth.

In another aspect of the present invention, there is a provided a method of suppressing mineralization and adhesion of teeth in the periodontal ligament, the method comprising:
taking the periodontal ligament cells from a patient; and
overexpressing RGD-CAP in the periodontal ligament cells taken above; and
transplanting the periodontal ligament cells having RGD-CAP expressed therein together with a tooth to be transplanted.

In this method, first, cells are taken from the periodontal ligament and maintained as described in Examples. The obtained cells were maintained in Dulbecco's modified Eagle's medium (DMEM; GIBCO, Grand Island, N.Y., USA) supplemented with 10% fetal calf serum (FCS; GIBCO), 100 units penicillin, and 100 μg/ml of streptomycin (GIBCO) (Medium A) in an atmosphere of 5% CO₂ in a humidified incubator.

RGD-CAP may be expressed in the periodontal ligament cells taken in accordance with a common method, more specifically, by introducing a mammalian expression vector containing a gene encoding RGD-CAP into the periodontal ligament cells. The expression vector can be introduced into mammalian cells by the method mentioned above. The expression vector is not limited as long as it expresses an inserted gene in mammalian cells, particularly, in human cells.

As a result that the expression vector is introduced, the periodontal ligament cells expresses RGD-GAP. The periodontal ligament cells having RGD-CAP expressed therein are then transplanted together with the tooth to be transplanted. The periodontal ligament cells to be transplanted may at least contain a mammalian expression vector containing a gene encoding at least RGD-CAP. The RGD-CAP used may not be expressed at the time of transplantation. In addition, at the time of transplantation, the agent of the present invention for suppressing mineralization of the periodontal ligament and preventing adhesion of teeth may be used.

According to another aspect of the present invention, there is a provided a method of suppressing mineralization in the periodontal ligament and preventing adhesion of teeth by applying RGD-CAP to the periodontal ligament when the tooth is transplanted.

For example, the method of the present invention is performed by applying RGD-CAP to the periodontal ligament when a tooth is transplanted to a patient. RGD-CAP may be directly applied to a tooth to be transplanted or applied to the site of the patient of the transplanting tooth. RGD-CAP may be used in the form of a composition containing RGD-CAP. More specifically, the agent for suppressing the mineralization of the periodontal ligament and preventing adhesion of teeth may be used.

All references cited are herein specifically incorporated by reference for all that is described therein.

### EXAMPLES

Materials & Methods

Proteins and Antibody

Recombinant human RGD-CAP was expressed by an expression vector pET-28a (Novagen, Madison, USA), and the inclusion bodies were obtained and purified as described previously (Ohno et al., 1999).

Briefly, Full-length cDNA (3041 bp) was isolated from a cDNA library of chick sternal cartilage and inserted in the EcoRI site of the expression vector pET-28a (Novagen, Madison, Wis., USA) to give an expression plasmid encording a peptide corresponding to amino acids 1-680 of RGD-CAP. The clone (pTE-CAP) containing the insert in the correct orientation was selected and sequenced to ensure that no mutations or deletions had occurred during the cloning procedure. E. coli strain HMS 174 (DE3) (Novagen) transformed with pTE-CAP was incubated while shaking in Luria-Bertani medium containing 30 μg/ml kanamycin at 37° C. until the optical density (OD) at 600 nm reached 0.6. The T7 lac promoter was then activated with 1 mM isopropyl-b-D-(−)-thiogalactopyra-noside (Wako Pure Chemical Industries Ltd., Osaka, Japan). And the cells were incubated at 37° C. for 3 h. The cells were harvested and the inclusion bodies obtained as described by Marston. The inclusion bodies were dissolved in 10 mM Tris-HCl buffer (pH 7.4) containing 7 M urea. This solution was applied to a His-Trap column (Pharmacia Fine Chemical, Uppsala, Sweden) which was equilibrated with 20 mM sodium phosphate buffer (pH 7.4) containing 7 M urea, 0.5 M NaCl and 10 mM imidazole. After the column was washed with 20 mM sodium phosphate buffer (pH 7.4) containing 7 M urea, 0.5 M NaCl and 10 mM imidazole, the recombinant protein was eluted with 20 mM sodium phosphate buffer (pH 7.4) containing 7 M urea, 0.5 M NaCl and 500 mM imidazole. The samples were dialyzed against 10 mM Tris-HCl buffer (pH 7.4) containing 7 M urea.

Monoclonal antibody against human RGD-CAP/βig-h3 was kindly provided by Dr. Fukushima (Japan Tobacco Corporation, Yokohama, Japan).

Preparation of Cells

Human PDL cells were obtained from healthy human teeth indicated for extraction inevitable for orthodontic treatment following the methods described in detail in a previous study (Somerman et al., 1989).

Briefly, PDL tissues attached to the middle third of the root were gently curetted and removed. The periodontal ligament tisse were rinsed with biopsy medium, cut into small pieces, dispersed on glass slides, placed in Leighton tubes, and incubated in the biopsy medium at 37° C. in a humidified atmosphere of 5% carbon dioxide-95% air. The following day, the medium was replaced with culture medium (DMEM supplement with 100 units/ml penicillin, 100 mg/ml streptomycin, 1.16 g/l glutamin, and 10% FCS. When the cells surrounding the tissue explants were confluent, they were transferred to 75-mm² tissue culture flasks by use of 0.08% trypsin/0.04% ethylenediamine-tetraacetic acid (EDTA), pH 7.2, and are designated first transfer cells.

Prior to the experiment, informed consent was obtained from all the patients regarding the extraction of their teeth. The experimental protocols were approved in advance by the Ethics Committee on Experimental Studies with Human Subjects, Hiroshima University Faculty of Dentistry. For all experiments, passage 4-5 cells were used.

Cultures were maintained in Dulbecco's modified Eagle's medium (DMEM; GIBCO, Grand Island, N.Y., USA) supplemented with 10% fetal calf serum (FCS; GIBCO), 100 units penicillin, and 100 μg/ml of streptomycin (GIBCO) (Medium A) in an atmosphere of 5% CO₂ in a humidified incubator.

For investigating the RGD-CAP mRNA level and ALP activity in the PDL cell cultures, the cells seeded on a 10-cm dish in Medium A containing 50 μg/ml ascorbic acid, were treated with 10⁻⁸ M dexamethasone (Dex) or 10⁻⁸ M 1α,25-dihydroxyvitamin D₃ (vitamin D₃) for 11 days after confluence.

For investigating the effects of RGD-CAP on the mineralization of PDL cells, the cells seeded on 35-mm dish were maintained in mineralizing medium (DMEM containing 10% FCS, 50 μg/ml ascorbic acid, 100 nM β-glycerophosphate and 10⁻⁸ M Dex) for 21 days.

Western Blot Analysis

RGD-CAP is so tightly attached to the insoluble collagen fibers that this protein is resistant to protease and homogenization (Hashimoto et al., 1997). Therefore, we solubilized the samples of human PDL in laemmli buffer in 4 M urea and boiled them for 10 min. Samples of 1 mg were separated by SDS-PAGE in a 4-20% poly acrylamide gradient gel, in the presence of β-mercaptoethanol (0-5.0%) that breaks aggregates stabilized by disulfide bonds. Proteins were blotted onto polyvinylidene difluoride membranes using a semidry electroblotter. After blocking, the membranes were incubated in phosphate-buffered saline (PBS, pH 7.4) containing anti-human RGD-CAP/βig-h3 monoclonal antibody overnight, and then in PBS containing ¹²⁵I-irradiated sheep anti-rat IgG (Fab′)₂ fragment (Amersham, Aylesburg, UK) for 3 hr at room temperature. The membrane was exposed to X-ray film. Polymerase Chain Reaction (PCR) analysis Total RNA was isolated from cultured PDL cells using a Total RNA Extraction Kit (Pharmacia Biotech Quick Prep^{R}, Tokyo, Japan) according to the manufacturer's instructions.

Single stranded cDNA was synthesized from 1 μg of total RNA using Oligo (dT)₂₀ primer (Toyobo, Osaka, Japan) and a Rever Tra Ace-α first strand cDNA synthesis kit (Toyobo).

Quantitative real-time polymerase chain reaction (PCR) was performed for examining the RGD-CAP mRNA level using an automated fluorometer (ABI Prism 7700 Sequence Detection System, PE Biosystems, Foster, USA), as described previously (Leutenegger C M, von Rechenberg B, Huder J B, Zlinsky K, Mislin C, Akens M K, et al. (1999). Quantitative real-time PCR for equine cytokine mRNA in nondecalcified bone tissue embedded in methyl methacrylate. Calcif Tissue Int 65: 378-383.).

Table 1(a) shows the sequences of the primers and probes for RGD-CAP and glyceraldehyde-3-phosphate dehydrogenase (G3PDH).

Comparative quantification of the RGD-CAP signals was performed by normalizing the RGD-CAP signals relative to those of G3PDH.

Reverse transcriptional (RT)-PCR was performed for examining the mRNA level of type I collagen and bone sialoprotein in cultured PDL cells maintained in mineralizing medium by use of a Gene AMP PCR system 2400 (Perkin-Elmer, Branchburg, USA). For the PCR reaction of bone sialoprotein, nested PCR primers were used. The pairs of degenerative primers were described in Table 1(b). The signal intensity-detected ethidium bromide was analyzed by scanning density (NIH image, version 1.59). We determined the relative mRNA expression of type I collagen or bone sialoprotein by dividing the densitometric value of RT-PCR products of each transcript by that of G3PDH.

Measurement of ALP Activity in Cultured PDL Cells

The PDL cells were washed three times with PBS, and 0.2 ml of 10 mM Tris-HCl containing 5 mM MgCl₂ was added. The cells were then sonicated for 1 min. The sonicates were centrifuged for 10 min at 3000 g, and the supernatants were used for the enzyme assay. ALP activity was assayed using p-nitrophenylphosphate as a substrate following the method described previously (Piche J E, Carnes D L, Jr., Graves D T (1989). Initial characterization of cells derived from human periodontia. J Dent Res 68: 761-767).

The amount of p-nitrophenol produced was measured spectrophotometrically at 410-nm and normalized by dividing the quantity by the cell number in each dish.

Alizarin Red Staining

Recombinant RGD-CAP (20 μg/ml) in the solution buffer (PBS containing 4 M urea) or solution buffer were added to the PDL cell cultures maintained in mineralizing medium every 2 days for 21 days. The cells were rinsed twice with PBS and incubated in 40 mM Alizarin red solution (Sigma, St. Louis, USA) at room temperature for 30 min. After washing twice with PBS, the cells were observed by light microscopy.

Results

Expression of RGD-CAP in the PDL

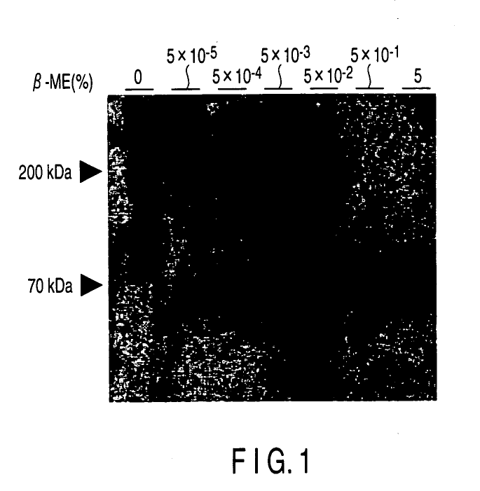

Western blot analysis in crude human PDL revealed that native RGD-CAP migrated as multiple bands of over 200-kDa. The bands were shifted to a single band corresponding to about 70-kDa under reducing conditions (in the presence of β-mercaptoethanol) (FIG. 1). RGD-CAP mRNA level and ALP activity in the PDL cell cultures

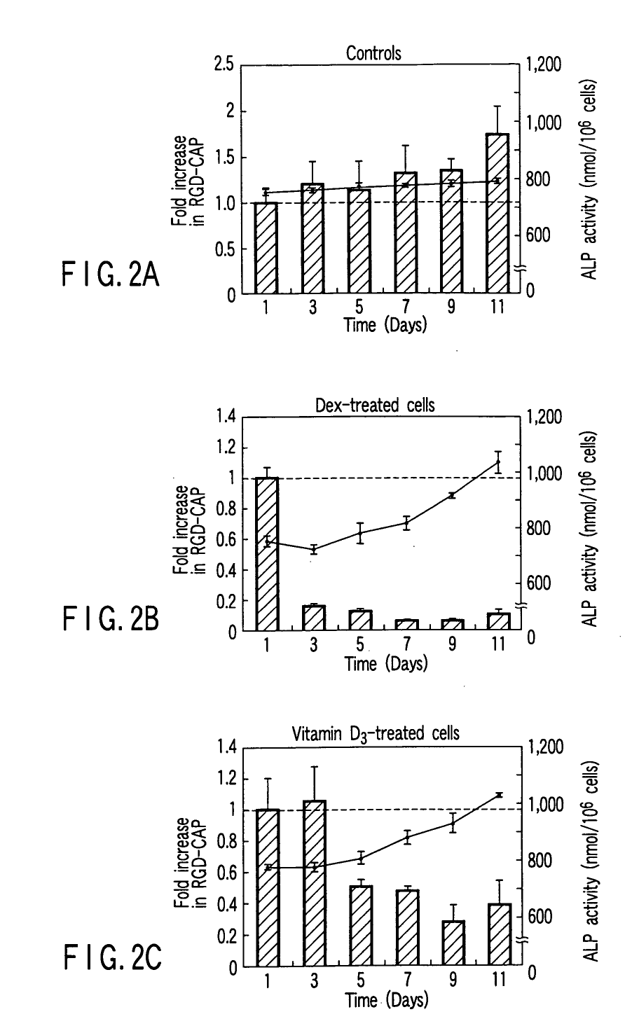

In the PDL cell cultures maintained in Medium A, ALP activity was not essentially changed through out the experimental period after the cells reached confluence. During this period, the mRNA level of RGD-CAP in the cultured cells increased gradually and reached about 1.7-times the basal level on day 11 (FIG. 2A). Treatment of the cells with 10⁻⁸ M Dex or 10⁻⁸ vitamin D₃ resulted in progressive increases in the ALP activity as compared to the controls. On the other hand, the mRNA level of RGD-CAP markedly decreased in the cultures after addition of Dex or vitamin D₃ (FIGS. 2B and 2C).

Effects of RGD-CAP on Mineralization of PDL Cells

The level of ALP activity was significantly lower in the PDL cell cultures on dishes coated with RGD-CAP (20 μg/ml) at 1-3 days after seeding as compared to the control dishes, but recovered to the control level at 5 days (FIG. 3A).

However, it may not say that RGD-CAP has the function of suppressing minelarization merely based on the fact that RGD-CAP can suppress the ALP activity. Then, to demonstrate whether RGD-CAP suppresses mineralization or not, mineralization markers other than the APL activity influenced by RGD-CAP were investigated.

The treatment of RGD-CAP on PDL cells maintained in mineralizing medium inhibited the decrease of type I collagen mRNA level, and resulted in the reduction of bone sialoprotein mRNA level (FIG. 3B). The treatment with recombinant RGD-CAP on cultured PDL cells maintained in mineralizing medium showed a decrease in the intensity of alizarin red staining and the bone nodule formation (FIG. 3C).

We demonstrated that recombinant RGD-CAP suppressed the ALP activity and bone nodule formation of cultured PDL cells. These results emphasize that RGD-CAP/βig-h3 contributes to the maintenance of the elasticity of PDL by inhibiting mineralization.

In conclusion, RGD-CAP may play an important role in the maintenance of PDL homeostasis by regulating the mineralization.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. An agent containing RGD-CAP for suppressing the mineralization in the periodontal ligament.

2. An agent containing RGD-CAP for preventing adhesion of teeth.

3. A method of suppressing the mineralization in the periodontal ligament and preventing the adhesion, the method comprising:
taking the periodontal ligament cells from a patient; and
overexpressing RGD-CAP in the periodontal ligament cells taken; and
transplanting the periodontal ligament cells having RGD-CAP expressed therein together with a tooth to be transplanted.

4. A method of suppressing mineralization and adhesion in the periodontal ligament by using RGD-CAP at the time of tooth transplantation.
